(19) United States
(12) Patent Application Publication
(10) Pub. No.: US 2017/0122535 A1
(43) Pub. Date: May 4, 2017

(54) COMPACT MOTION-ACTIVATED UTILITY LIGHT WITH ADJUSTABLE ARM

(71) Applicants: Matthew Alexander, Provo, UT (US); Michael Kannely, Provo, UT (US)

(72) Inventors: Matthew Alexander, Provo, UT (US); Michael Kannely, Provo, UT (US)

(21) Appl. No.: 14/928,942

(22) Filed: Oct. 30, 2015

(51) Int. Cl. H01J 40/14; H05B 37/02; F21L 4/00; F21V 33/00; A61L 9/14; F21V 21/28; F21V 21/092

(52) U.S. Cl. CPC F21V 21/28; H05B 37/0227; F21L 4/00; F21V 21/092; H05B 37/0218; F21V 33/004; A61L 9/14; A61L 2209/111; A61L 2209/12

(57) ABSTRACT

A compact motion-activated light with flexible arm that enables a user to illuminate remote areas that would otherwise be difficult or impossible to illuminate. The device contains its own power source so that it does not need to be connected to an electrical outlet, enabling the user to place the device in areas where there are not electrical outlets or even no wired electricity such as basements, attics or garages.

### CROSS REFERENCE TO RELATED APPLICATION

This application claims the benefit of U.S. Provisional Application Ser. No. 62/111,081 filed Feb. 4, 2015 by Matthew Aaron Alexander; U.S. Provisional Application Ser. No. 62/117,448 filed Feb. 15, 2015 by Matthew Aaron Alexander; U.S. Provisional Application Ser. No. 62/117,453 filed Feb. 15, 2015 by Matthew Aaron Alexander; and U.S. Provisional Application Ser. No. 62/131,571 filed Mar. 11, 2015 by Matthew Aaron Alexander.

### FIELD OF THE INVENTION

The present invention relates generally to utility lights and more particularly to a compact motion-activated light with flexible arm for use in remote locations.

### BACKGROUND OF THE INVENTION

One problem that is universally faced by almost all people is being able to find their way or see things in the dark. It happens when a person wakes up in the middle of the night. It happens when a person goes into a basement. It happens when a person goes into a garage or attic. It happens when a person is outside at night.

Conventionally, illumination is provided by attached and/or wired lighting fixtures, such as ceiling lights or lamps. People have also used small nightlights plugged into electrical outlets to provide illumination. If a person needs light upon waking in the middle of the night, however, they may be disoriented and then suddenly shocked when they turn on a light switch, which turns on a bright light to which their eyes have difficulty adjusting. Similarly, when a person turns off a bright light, their eyes have problems adjusting to the dark, and they have a hard time seeing. This also holds true when a person is looking in the dark corners of their basement or garage. Additionally, it is often impossible to provide lighting fixtures in these locations because of a lack of electrical outlets.

This problem is particularly pronounced when a person wakes up to use a restroom during the night. They do not want to fumble with a light switch or be blinded when they are able to turn a light on. Others have addressed this problem with motion activated lights that turn on when a person enters a dark space. Specifically, there are lights that hook over the edge of a toilet bowl and activate when person enters the darkened restroom. But these lights require that the whole light assembly of the device and method of attaching the device be exposed to the toilet bowl and often require that a bulky housing be located along with the light.

When a light is needed in a remote location, there is often not enough room to place any kind of a lighting device let alone one that is bulky or requires that it be connected to an electrical outlet. In addition, even if the device is wireless, the lighting element of known devices is either a part of the device housing or rigidly attached to the device housing. Therefore, if a person wants to provide a light in a remote location their options are limited.

The present invention provides a solution to shortcomings of existing devices. It provides a compact motion-activated light with adjustable arm that allows the entire unit and its lighting element to be placed in remote locations and inserted to illuminate areas where conventional lighting cannot be installed or utilized. The adjustable arm allows for an infinite array of lengths and angles for lighting so that custom locations can be illuminated where the rest of the body of the device cannot or should not fit.

### SUMMARY OF THE INVENTION

The present invention has been accomplished to provide a compact motion-activated utility light with adjustable arm.

According to the present invention, there is provided a compact motion-activated utility light with adjustable arm, which is generally comprised of a housing, a motion sensor, a light sensor, a power source, and adjustable arm and a light.

In one aspect of the invention, it provides a light for a toilet bowl to allow a user to find their way in the dark without turning on bright light fixtures.

In another aspect of the invention, it provides an ultraviolet light to disinfect an area by killing microorganisms by destroying nucleic acids and disrupting their DNA.

In another aspect of the invention, it provides a light to be placed in hard to reach and unconventional locations and custom fit to illuminate such locations.

In another aspect of the invention, it provides a light that can be placed using the adjustable arm to illuminate an area where the unit that provides power to the light cannot be located.

To the accomplishment of the above and related aspects, the invention may be embodied in the form illustrated in the accompanying drawings. The drawings, however, are illustrative only. Variations are contemplated as being part of the invention, limited only by the scope of the claims.

### BRIEF DESCRIPTION OF THE DRAWINGS

FIG. 1 is a front view of the present invention.

FIG. 2 is a side view of the present invention.

FIG. 3 is a back view of the present invention.

### DETAILED DESCRIPTION OF THE INVENTION

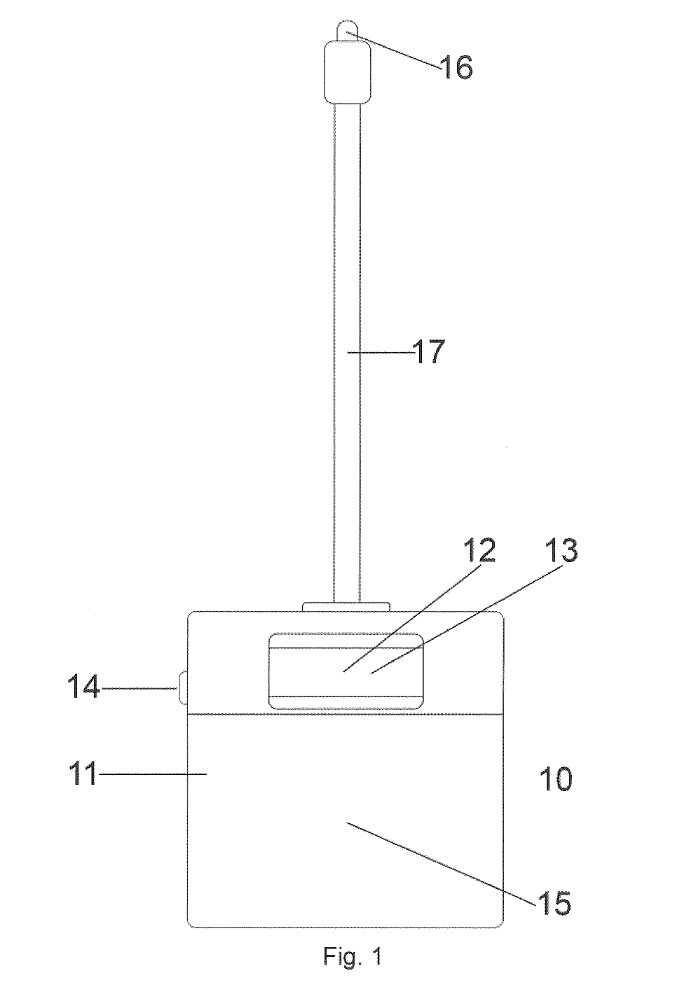

As shown in the accompanying drawings, this is a compact motion-activated utility light with adjustable arm. FIG. 1 shows that compact motion-activated utility light with adjustable arm 10 includes a body member 11, which includes motion sensor 12, light sensor 13, settings button 14 and battery compartment 15. Light 16 is attached to body member 11 by adjustable arm 17. Adjustable arm 17 can be used to adjust light's 16 distance from body member 11 by being extended away from body member 11 or drawn closer to body member 11 by collapsing adjustable arm 17 upon itself or within a cavity in body member 11. While the present embodiment includes light sensor 13, which is used to ensure light 16 only activates when it is dark, such as when it is being used to illuminate a toilet at night or when it is being used outdoors, light sensor 13 may not be present or be deactivated when compact motion-activated utility light with adjustable arm 10 is being used in a location that is usually dark such as remote areas in a basement, attic or garage.

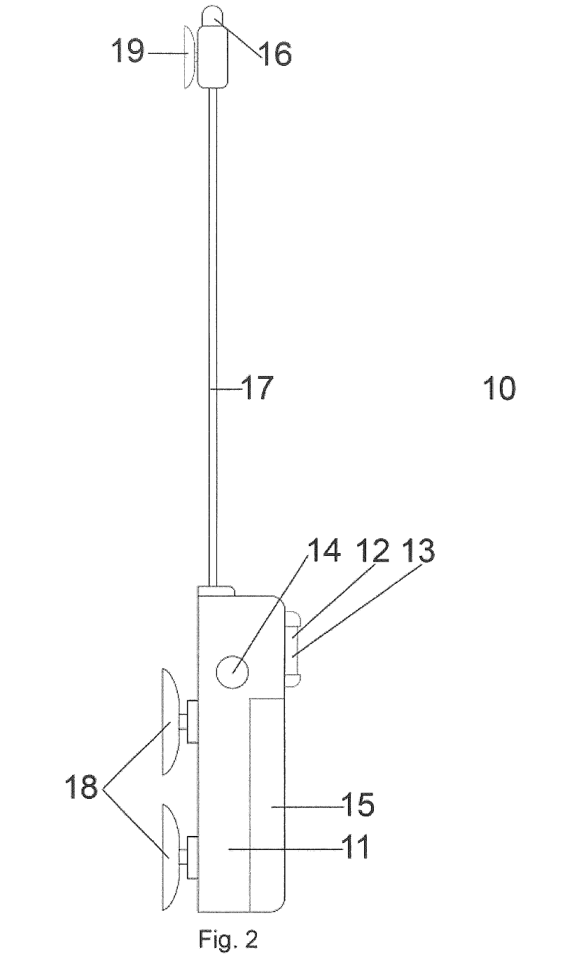

FIG. 2 shows compact motion-activated utility light with adjustable arm 10 to include body member 11, motion sensor 12, light sensor 13, settings button 14, battery compartment 15, light 16 and adjustable arm 17. In addition FIG. 2 shows body member suction cups 18 and light suction cup 19. It is understood that other attachment means, including but not limited to, glue, tape, stickers, nails or screws, could be used in place of body member suction cups 18 and light suction cup 19.

Settings button 14 can be used to allow the user to select different color for light 16, especially if light 16 is an LED light. Light 16 can be any kind of a light, including but not limited to an LED light, a compact fluorescent light or a UV light. Settings button 14 can also be used to allow the user to select between an LED light or a UV light in the event that the user wants to use compact motion-activated utility light with adjustable arm in a disinfecting mode. Settings button 14 can also be used to turn off power to compact motion-activated utility light with adjustable arm 10. Battery compartment 15 provides a location for a power source that is used to power compact motion-activated utility light with adjustable arm 10. It is understood that the power source could be a battery but could also include a solar cell or other power generating apparatus.

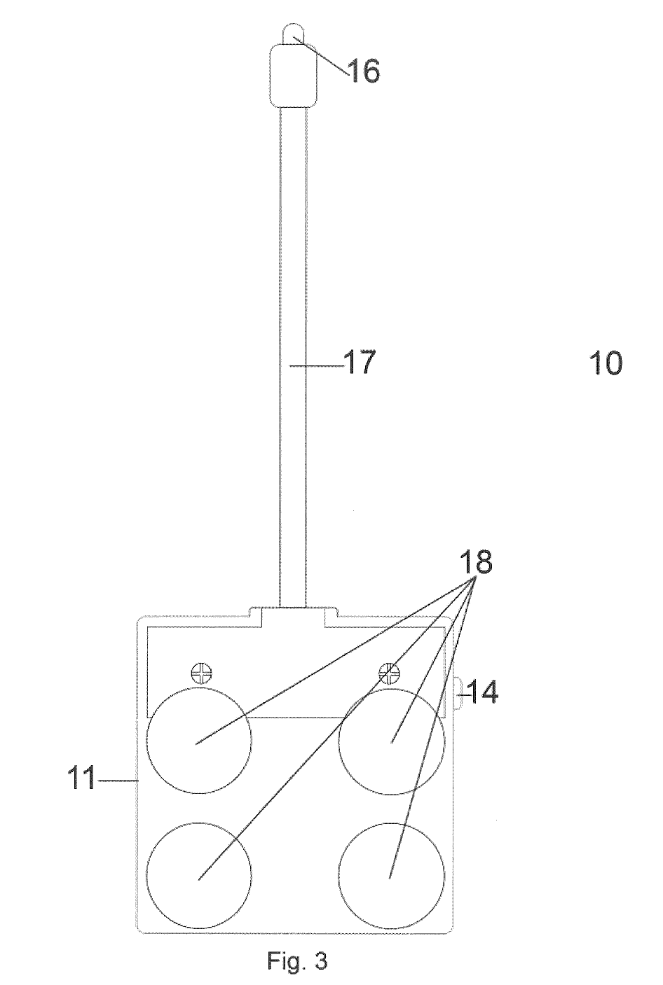

FIG. 3 shows the back of compact motion-activated utility light with adjustable arm 10, including body member 11, settings button 14, light 16, adjustable arm 17 and body member suction cups 18. In this configuration, compact motion-activated utility light with adjustable arm 10 can be easily attached in a remote location, such as the side of a toilet bowl or the corner of a basement using body member suction cups 18. Light 16 can then be placed wherever the user desires by utilizing adjustable arm 17. Adjustable arm 17 does not have any impact on body member 11 being attached to a location. Only body member suction cups 18 or other attachment means located on body member 11 are used to attach body member 11. The very nature of adjustable arm 17 means that it does not provide any structural support for attaching body member 11 to a location. Adjustable arm 17 is made of material that transports light such as fiber optics, light pipe or other light transporting materials. Adjustable arm 17 can be bent and formed any way to fit any location, allowing the thin flexible arm to be positioned out of the way to allow illumination to a remote area.

If being used to illuminate a toilet bowl, compact motion-activated utility light with adjustable arm 10, could also be configured to include means for dispersing other substances apart from lights, including but not limited to perfumes or germicidal sprays.

The description of the invention above should not be interpreted as limiting the invention to the disclosed embodiment because those who are skilled in the art to which the invention relates will be able to devise other equivalent forms thereof within the scope of the invention. Variations and changes, which are obvious to one skilled in the art, are intended to be within the scope and nature of the present invention.

What is claimed is:

1. A compact motion-activated utility light with adjustable arm, comprising:
a body member;
a light;
a power source;
at least one adjustable arm connecting said light to said body member;
a motion detector situated on said body member and directed away from said body member so as to detect motion a distance away from said body member and configured to activate said power source when motion is detected;
wherein said power source is positioned in said body member and configured to energize said light when said motion detector senses motion.

2. The compact motion-activated utility light with adjustable arm of claim 1 further comprising a light detector situated on said body member and directed away from said body member so as to detect ambient light levels and configured to active said power source when ambient light falls below a desire level.

3. The compact motion-activated utility light with adjustable arm of claim 1 wherein said light is a light emitting diode.

4. The compact motion-activated utility light with adjustable arm of claim 2 wherein said light is a light emitting diode.

5. The compact motion-activated utility light with adjustable arm of claim 1 wherein said light is a short wavelength ultraviolet light.

6. The compact motion-activated utility light with adjustable arm of claim 2 wherein said light is a short wavelength ultraviolet light.

7. The compact motion-activated utility light with adjustable arm of claim 1 further comprising a timer that de-energizes the power source after a predetermined amount of time.

8. The compact motion-activated utility light with adjustable arm of claim 2 further comprising a timer that de-energizes the power source after a predetermined amount of time.

9. The compact motion-activated utility light with adjustable arm of claim 7 further comprising a dispenser that disperses a substance to freshen the air when the power source de-energizes.

10. The compact motion-activated utility light with adjustable arm of claim 8 further comprising a dispenser that disperses a substance to freshen the air when the power source de-energizes.
